(19) United States
(12) Patent Application Publication
(10) Pub. No.: US 2022/0143430 A1
(43) Pub. Date: May 12, 2022

(54) DEVICE FOR PRODUCING AN AIR CURTAIN IN FRONT OF A FACE OF A USER

(71) Applicant: Hilti Aktiengesellschaft, Schaan (LI)

(72) Inventors: Stefan KRAEMER, Augsburg (DE); Lars MELZER, Hohenfurch (DE)

(21) Appl. No.: 17/437,497

(22) PCT Filed: Mar. 23, 2020
(86) PCT No.: PCT/EP2020/057952

§ 371 Date: Sep. 9, 2021

(30) Foreign Application Priority Data
Apr. 4, 2019 (EP) 19167199.9

(51) Int. Cl. A62B 18/00; A62B 7/10; A62B 18/04; A61F 9/02

(52) U.S. Cl. CPC A62B 18/003; A61F 9/029; A62B 18/04; A62B 7/10

(57) ABSTRACT

A device for producing an air curtain in front of a face of a user, wherein the device includes, inter alia, a pump for supplying air to produce the air curtain and at least one nozzle for forming the air curtain.

The present invention relates to a device for producing an air curtain in front of a face of a user, wherein the device comprises, inter alia, a pump for supplying air to produce the air curtain and at least one nozzle for forming the air curtain.

### BACKGROUND OF THE INVENTION

Devices for protecting workers, e.g. in coal mines, or construction workers on construction sites, from breathing in contaminated air are known in the prior art. In coal mines or on construction sites, the breathable air available for the workers may be contaminated, particularly with dust, pollutants or noxious particles. Conventional protection devices that are known from the prior art can be, for example, protective helmets that can be placed on the shoulders of the worker to prevent the dust particles or noxious particles getting into the region of the nose of the worker. These protection devices, which are often designed in the form of hoods, are generally closed—in a manner similar to a diver's helmet. The disadvantage with these devices is that they are often uncomfortable to wear and are therefore frequently not accepted by the workers. Moreover, the mobility and field of view of the worker may be restricted by such closed hoods.

### SUMMARY OF THE INVENTION

It is an object of the present invention to overcome the above-described disadvantages of conventional protection devices and to provide a device which ensures effective protection for the worker without restricting their mobility and field of view. In addition, the device to be provided should be as light and compact as possible, ensuring that it has the maximum possible wearing comfort and is well accepted by the workers. Furthermore, it should be possible to wear the device to be provided, irrespective of whether the user has a beard or wears spectacles to improve vision. Another concern of the invention is to affect the inhalation resistance of the user as little as possible, ensuring that inhalation by the worker is as far as possible not made more difficult by wearing the device to be provided.

The present invention provides a device for producing an air curtain in front of a face of a user. The device is characterized in that the device comprises the following components:

at least one pump for supplying air to produce the air curtain,

at least one nozzle for forming the air curtain,

wherein the at least one nozzle is arranged in the region of a nose of the user on a supporting device in such a way that a mouth-nose region of the user is covered by the air curtain. According to the invention, the device is preferably also referred to as a “protection device”.

It was completely surprising that it is possible to provide a protection device with which the user of the device can inhale through the air curtain, wherein the dust particles and/or noxious particles are deflected by the curtain, with the result that as far as possible the user does not inhale any dust and pollutants from the atmosphere. This advantage is achieved, in particular, through the clever arrangement of the at least one nozzle in relation to a face of the user. In particular, the proposed arrangement of the at least one nozzle advantageously enables the entire mouth-nose region of the user to be covered by the air curtain. According to the invention, the term “mouth-nose region of the user” describes the region of the face of a user which extends between the mouth and the nose of the user and includes the mouth of the user. The mouth-nose region is preferably of substantially triangular form, wherein the mouth defines a lower side of the mouth-nose region, and the nose defines the tip of the mouth-nose region of substantially triangular form. The mouth-nose region is preferably larger than the area between the mouth and the nose. In other words, the mouth-nose region overlaps the actual area between the mouth and the nose and extends upward as far as the tip of the nose, for example. On the lower side of the mouth-nose region, which is preferably triangular in form, the mouth-nose region projects downward beyond the mouth and to the right and left, e.g. by a length of 2 cm in each case.

The air curtain too, which is formed by the at least one nozzle, is preferably of substantially triangular design, wherein the shape of the air curtain is characterized by an opening angle gamma in the region of the nozzle of the device. This opening angle can be in a range of 90 degrees, for example, wherein the opening angle is measured between two outer air or edge jets of the air curtain. An opening angle gamma/2 is preferably defined between a virtual axis extending centrally through the face of the user and one of the two outer air jets of the air curtain, wherein half the opening angle gamma/2 is preferably in a range of from 0 to 45 degrees.

The air curtain is preferably formed by an air flow produced by the pump of the protection device. According to the invention, the term “air” denotes a gas mixture, which, in particular, includes nitrogen N2 and oxygen O2 as well as various further gases and gas mixtures. The air is preferably drawn in by the pump from the surroundings of the user and is passed to the nozzle via conduit means, e.g. an air hose. In a preferred embodiment of the invention, the supporting device is a protective helmet or safety goggles. If the supporting device of the protection device is designed as safety goggles or as a protective helmet, the pump is preferably arranged on a rear side of the protection device, which is preferably arranged on the back of the head of the user. The supporting device preferably forms the basic structure for the protection device, wherein the various components of the protection device can be secured on the supporting device.

The pump is preferably designed to deliver and output air substantially without pulsations, with the result that a laminar flow that forms the air curtain is advantageously formed. In respect of the volume flow that is required to produce the air curtain, the pump is preferably matched to the at least one nozzle or the nozzle assembly or the geometry thereof. In other words, the volume flow of the pump can be designed to match the nozzle or the nozzle assembly. According to the invention, it is very particularly preferred that the pump is designed to overcome the vacuum which prevails in a filter of the protection device. In particular, it is preferred according to the invention that the pump ensures a substantially pulsation-free air flow by means of its principle of operation. For example, it is possible to use piezoelectric pumps that operate at frequencies in a range of from 30 to 40 kHz, preferably at a frequency of about 35 kHz. Of course, it is also possible to use pumps or piezoelectric pumps which operate in other frequency ranges. In particular, frequencies greater than 500 Hz, preferably: f>500 Hz, are in accordance with the invention. For example, it is possible to use pumps that operate at frequencies of 1000 Hz=1 kHz, 2 kHZ, 3 kHz, 4 kHz, 5 kHz, 10 kHz, 15 kHz, 20 kHz, 25 kHz or 45 kHz, 50 kHz, 55 kHz, 60 kHz etc. Of course, all intermediate values are also preferred. In particular, frequencies of the piezoelectric pump in a range of from 500 Hz to 500 kHz, preferably in a range of from 1 kHz to 100 kHz, particularly preferably in a range of from 10 kHz to 80 kHz and most preferably in a range of from 30 to 40 kHz, may be preferred. Moreover, it is possible to use rotary slide-valve pumps, which likewise operate substantially without pulsations.

For the provision of a laminar air flow, it is preferred that a speed within the air flow that is not too high is selected in order to prevent formation of vortices and turbulent flows. According to the invention, it is preferred that the nozzle is matched to the volume flow of the pump. According to the invention, it is furthermore preferred that the pump is operated at a volume flow in a range of from 1.0 to 1.5 l/min, a volume flow of 1.2l/min being particularly preferred. This preferred volume flow can preferably be combined with a flow rate in a range of 1-2 m/s. By means of this combination of flow rate and volume flow, a laminar air flow is advantageously achieved. According to the invention, it is preferred, in particular, that a nozzle geometry is designed in such a way that the air flow is spread out as widely as possible.

The pump is preferably used to supply air to produce the air curtain. For this purpose, it is possible, in particular, to use rotary slide-valve pumps or piezoelectric pumps. Piezoelectric pumps especially are particularly inexpensive and have a low weight. According to the invention, it is preferred that the user of the protection device, that is to say, for example, the wearer of the safety goggles or the protective helmet, breathes through the air curtain. The fine dust particles or noxious particles are preferably deflected by the preferably fast flowing air curtain. According to the invention, it is preferred that a speed of the air flow or air curtain is about 2 m/s in the region in front of the nose of the user of the protection device and about 1 m/s in the region in front of the mouth of the user. The flow rate preferably depends on the speed at which at least a portion of the air flow or of the air curtain is drawn in by the mouth or nose of the user. By virtue of the different flow cross sections of the mouth and nose, different flow rates can be produced in the air flow or in the air curtain in front of the mouth or in front of the nose of the user.

It is a particular advantage of the proposed protection device that the air does not encounter any resistance in the context of the present invention, thus ensuring that the inhalation resistance of the user of the protection device is not increased. In other words, it is preferred according to the invention that the wearer of the safety goggles or of the protective helmet can inhale and exhale equally or at least similarly well when using the protection device as without a protection device. The safety goggles or the protective helmet advantageously prevent the penetration of dust or noxious particles from above.

The air curtain preferably covers a large region of the lower half of the face of the user. In this context, a mouth-nose region of the user can be defined that preferably has a triangular basic shape and is slightly larger than the area defined by the mouth and nose. The protection device preferably ensures coverage of the mouth-nose region with the air curtain, irrespective of whether there are external, possibly disruptive, air flows or movements. In particular, optimum coverage of this kind can be made possible by the synergistic interplay between the pump and at least one nozzle or nozzle assembly. It is a particular merit of the invention that the protection device prevents the inhalation of dust particles and noxious particles particularly effectively, irrespective of whether the wearer of the safety goggles or of the protective helmet has a beard and/or is wearing spectacles.

The air curtain is preferably formed by the at least one nozzle. According to the invention, an air flow which is defined by an opening angle gamma is particularly preferred. The shape of the air curtain can be determined, in particular, by the geometry of the at least one nozzle. According to the invention, there can also be a preference for the function of the nozzle to be carried out by a nozzle assembly. The nozzle assembly can preferably comprise two or more nozzles, wherein the individual nozzles are, in particular, arranged in the region of a nose of the user of the protection device. Preferred nozzle assemblies are illustrated, in particular, in the attached figures. In order to shape the air flow into an air curtain, other components of the protection device can also make a contribution. For example, the flow can be passed over components of safety goggles if the supporting device is formed by safety goggles. The nozzle or nozzle assembly is preferably designed to produce or shape a laminar flow to produce the air curtain. It has advantageously been found that the nozzles operate with particularly low losses in the context of the present invention. The nozzle or nozzle assembly advantageously makes possible substantially complete coverage of the lower half of the face or of the mouth-nose region of the user of the protection device. This substantially complete coverage is made possible, in particular, by the shape of the air curtain provided by the nozzle or the nozzle assembly, the shape being decisively influenced by the positioning of the nozzles or of the assembly of nozzles within the protection device. In a particularly preferred embodiment of the invention, two nozzles can be provided on the protection device, wherein the two nozzles are arranged on the right and the left below safety goggles integrated into a helmet of the protection device.

According to the invention, it is preferred that the at least one nozzle or nozzle assembly is at a distance d from a face of the user, wherein the distance d is in a range of 2 to 30 mm, preferably 5 to 20 mm and particularly preferably 10 mm. In other words, it is preferred according to the invention that there is a distance between the nozzle or nozzle assembly and the nose, in particular the tip of the nose, of a user. In a very particularly preferred embodiment of the invention, this distance is about 1 cm. The term “distance” preferably denotes a minimum distance between the tip of the nose of the user of the protection device and the nozzle or nozzle assembly. Swirling is advantageously minimized by means of the preferred distance, as tests have shown.

The at least one nozzle or nozzle assembly is preferably designed to emit an air flow at an angle alpha, wherein the angle alpha is in a range of from 10 to 30 degrees. According to the invention, it is preferred to set the angle alpha in such a way that no air gap is formed at the chin of the user. The angle alpha is preferably measured starting from a vertical axis. This can preferably be the vertical virtual axis, which preferably extends centrally through the face of the user, divides the face of the user notionally into two halves and is used in defining the opening angle gamma/2, for example. According to the invention, it is preferred that the nozzle or nozzle assembly slopes in the direction of the face of the user. In other words, the nozzle or nozzle assembly of the protection device can be directed toward a chest or stomach of the user, with the result that the air flow produced, which forms the air curtain, extends at a distance of 1 to 3 cm in front of a chin of the user. A preferred course of the air flow, and the distance d and slope angle alpha are illustrated in FIG. 1, for example.

In combination with the abovementioned distance between the nozzle or nozzle assembly and the tip of the nose of the user, the setting of the angle alpha leads to optimum swirling behavior within the air flow. According to the invention, this preferably means that swirling can be minimized if a distance d is 1 cm, for example, and the angle alpha is set to between 10 and 30 degrees. Said angles and distances can be selected and/or varied, in particular, in accordance with the shape of the head of a user of the protection device.

According to the invention, it is preferred that the device comprises two nozzles, which preferably form a nozzle assembly. The nozzles preferably enclose an angle beta with one another, wherein the angle beta is in a range of from 0 to 10 degrees. In particular, the absolute value of the angle in relation to the two nozzles is in a range of from 0 to 10 degrees, wherein the signs of the slope angles of the two nozzles are preferably opposed. The air curtain, which preferably covers the entire nose-mouth region of the user, is achieved, in particular, through the clever arrangement of the nozzle assembly in relation to a face of the user. This assembly is characterized, in particular, by the angles alpha, beta and delta. According to the invention, it is preferred that the nozzles are of sloping design in relation to the vertical virtual axis, which preferably extends centrally through the face of the user and divides the face of the user notionally into two halves. The nozzles are preferably arranged on the right and left of this vertical axis, wherein the nozzles are of sloping design, in particular in a front or frontal view of the face of the user or of the invention. The two nozzles are preferably tilted in relation to the virtual vertical axis, wherein the nozzles can preferably be tilted in the manner illustrated in FIG. 2. In particular, a first nozzle can be arranged in front of a left-hand half of the face of the user, while a second nozzle of the nozzle assembly is arranged in front of a right-hand half of the face of the user. The terms “right” and “left” preferably relate to the virtual vertical axis corresponding to a front view of a face of a user of the protection device. Such a front view of the invention is shown in FIG. 2, for example. The terms “top” and “bottom” in the context of the present invention can preferably be interpreted to mean that the chin forms a lower region of the face of the user, while the upper part of the face is generally covered by hair. The two nozzles of the nozzle assembly in this embodiment of the invention are preferably at a shorter distance from the virtual vertical axis in a lower region than in the upper region of the nozzles. In other words, the two nozzles are arranged like the arms of a letter “V” relative to one another. The slope angle beta therefore preferably has a positive sign for one of the two nozzles, while the slope angle beta has a negative sign for the other nozzle of the nozzle assembly. The absolute values of the slope angle are preferably the same or substantially the same for the first nozzle and for the second nozzle.

Each of the two nozzles of the nozzle assembly preferably produces its own air curtain. The two separate individual air curtains are preferably superimposed to form an overall air curtain, which preferably covers the entire mouth-nose space of the user of the protection device. Through the provision of the two nozzles, which each enclose an angle beta with a virtual vertical axis, an overlap between the air curtains is achieved, preferably in the center of the face of the user. Thus, the overall air curtain formed or the underlying air flow is formed to a particularly pronounced degree in the region below the nostrils. The dust particles and noxious particles are thereby deflected and removed from the region below the nose in a particularly effective manner, thus preventing them from getting into the nose and airways of the user. The two nozzles preferably define an air curtain with an opening angle of gamma less than or equal to 90 degrees. By means of this opening angle, complete coverage of the mouth and of the region below the nose of a user by the overall air curtain is advantageously achieved.

According to the invention, it is preferred that the nozzles enclose an angle delta with a vertical plane, wherein the angle delta is in a range of from 10 to 30 degrees. The vertical plane can preferably comprise the vertical virtual axis, which preferably extends centrally through the face of the user, and can be formed substantially parallel to a plane of the face of the user. The sloping nozzles can preferably follow the course of the alae of the nose, as illustrated in FIG. 5. The angle delta is preferably formed between the nozzles and the vertical plane, wherein the absolute values of the slope angles of the first and the second nozzle are substantially the same, while the signs of the slope angles of the two nozzles are preferably opposed. The nozzles are preferably also arranged in a v shape in a top view from above (cf. FIG. 5), wherein the opening of the letter “V” points in the direction of the face of the user, while the point of the letter “V” faces away from the user. By means of the oblique positioning of the nozzle, improved coverage of the face with the air curtain is achieved, in particular. It is thereby advantageously avoided that dust particles can be inhaled by the user, bypassing the curtain.

The device preferably comprises a filter for cleaning the air which is drawn in by the protection device in order to produce the air flow to produce the air curtain. During the use of the protection device, it may happen that some of the air curtain is inhaled by the user. In order to ensure that no dust particles or noxious particles are inhaled during this process, it is preferred according to the invention that the air drawn in is filtered. The filter of the protection device is preferably arranged on a rear side of the device, and therefore the filter is arranged in the region of the back of the head of the user. The provision of the filter advantageously also protects the pump of the protection device and the mechanism thereof from contamination and damage. According to the invention, the use of filters of filter class H13 or HEPA may be preferred. The working time may be extended with the filter if a filter with a high filtration efficiency is used.

According to the invention, it is preferred that the protection device comprises a unit for monitoring a function of the device. According to the invention, this unit is preferably also referred to as a function monitoring unit. According to the invention, it is preferred that the function monitoring unit operates electronically. The function monitoring unit can preferably be a component of a control unit of the proposed protection device. The function monitoring unit preferably meets the demands on a warning device that is in some cases legally required for determining that a respirator or the protection device is failing or is operating incorrectly. The function monitoring unit can preferably carry out volume flow monitoring, wherein such volume flow monitoring may be difficult in combination with the air curtain. According to the invention, it is therefore very particularly preferred that monitoring of a differential pressure between the filter housing and the environment or the surroundings of the user is carried out. In other words, the function monitoring unit is designed to monitor a differential pressure between the protection device and the surroundings of the user. The function monitoring unit can furthermore preferably be used to monitor a filter state of the filter of the protection device.

In the context of the proposed function monitoring, it is preferred according to the invention that the function monitoring unit is capable of characterizing or detecting the filter used. Provision is furthermore made for the function monitoring unit to be able to detect a pressure drop across the filter in an unused and possibly a clogged or blocked state. In particular, the function monitoring unit has means by means of which the values determined can be compared with one another. In the case where a pressure difference is measured that is between the value “zero” and the differential pressure value measured for a new, unused filter, for example, it may be that there is a fault in the pump and/or in the power supply. Such a measurement result can also be interpreted to mean that there is a leak in the protection device or in the conduit means thereof. If a determined pressure difference is between a first value measured for a new, unused filter and a second value measured for a clogged state, it is possible to assume substantially fault-free functioning of the protection device. If the value for the differential pressure is above a certain limit value, it may be that the filter is clogged and should be replaced. In order to measure the differential pressure in the context of the proposed function monitoring, it is possible, in particular, to use differential pressure sensors.

The protection device preferably comprises an energy supply device for supplying energy for the components of the device. This can preferably be a current and/or voltage source. The energy supply device is preferably designed to supply the protection device with sufficient electric energy for the operation of the components, e.g. the pump, the nozzles, the filter and/or the function monitoring unit. The energy supply device can be formed by a battery or by one or more storage batteries, for example. In the context of the present invention, it is possible to use 12 V storage batteries, for example.

It has been found that the nozzle or nozzle assembly is particularly significant for the functioning of the protection device, that is to say, in particular, the deflecting effect of the air curtain in relation to dust particles or noxious particles. The filter of the protection device can preferably be positioned on the back of the head of the user. This is associated with the advantage that the air at the back of the head of the user is often cleaner than the air in front of the face of the user, wherein this region often coincides with the work zone of the user. The function monitoring unit preferably operates on the basis of a differential pressure measurement. It can be arranged in a filter box within the protection device, for example. It is preferred in the sense that the energy supply device is arranged as deep as possible within the protection device. Optimum distribution of the weight of the energy supply device is thereby achieved, especially if the energy supply device is formed by storage batteries. The protection device can preferably also comprise a power supply device, which can be coupled to the pump of the protection device, for example. The power supply device and/or the pump of the protection device are preferably likewise arranged as deep as possible within the device. In one embodiment of the invention, it may be preferred that the power supply device and/or the pump are arranged for the purpose of balancing out the weight in relation to the storage batteries.

According to the invention, there is a particular preference for the protection device to have means for converting the direct voltage that is preferably output by the storage batteries into an alternating voltage that is required for the operation of piezoelectric pumps. The power supply device of the protection device is preferably designed to modify the voltage output by the storage battery in such a way that it can be used to operate the piezoelectric pumps if piezoelectric pumps are used as pumps.

Further advantages will become apparent from the following description of the figures. The figures, the description and the claims contain numerous features in combination. A person skilled in the art will expediently also consider the features individually and combine them to form expedient further combinations.

### BRIEF DESCRIPTION OF THE DRAWINGS

In the figures, identical components and components of identical type are designated by the same reference signs. In the figures:

FIG. 1 shows a side view of a preferred configuration of the invention

FIG. 2 shows a front view of a preferred configuration of the invention

FIG. 3 shows a schematic side view of a preferred configuration of the invention

FIG. 4 shows a schematic side view of a preferred configuration of the invention

FIG. 5 shows a plan view of a preferred configuration of the invention

### DETAILED DESCRIPTION

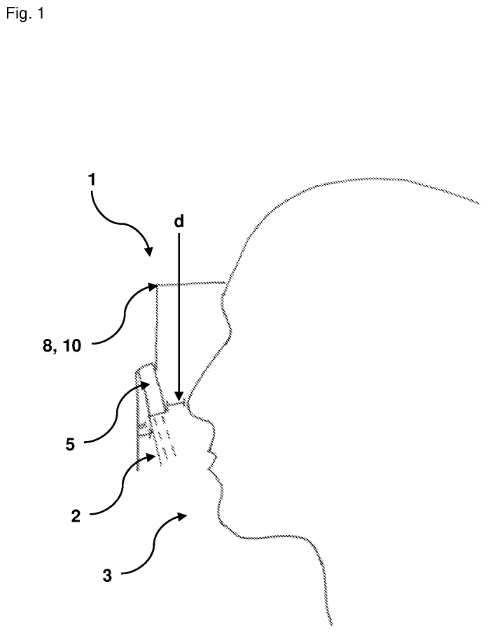

FIG. 1 shows a preferred configuration of a protection device (1). It illustrates a supporting device (10), which, in particular, can be designed as safety goggles (8). The supporting device (10) supports a nozzle (5) or a nozzle assembly (5), wherein the nozzle (5) or the nozzle assembly (5) is designed to form an air curtain (2) in front of the face (3) of a user of the protection device (1). The nozzle (5) or the nozzle assembly (5) is preferably of sloping design, such that it is at a shorter distance d from the face (3) of the user in a lower region than in an upper region. In other words, the lower region of the nozzle (5) or the nozzle assembly (5) is tilted in the direction of the face (3) of the user. This tilting or slope of the nozzle (5) or of the nozzle assembly (5) is described by the slope angle alpha, which is preferably in a range of from 10 to 30 degrees.

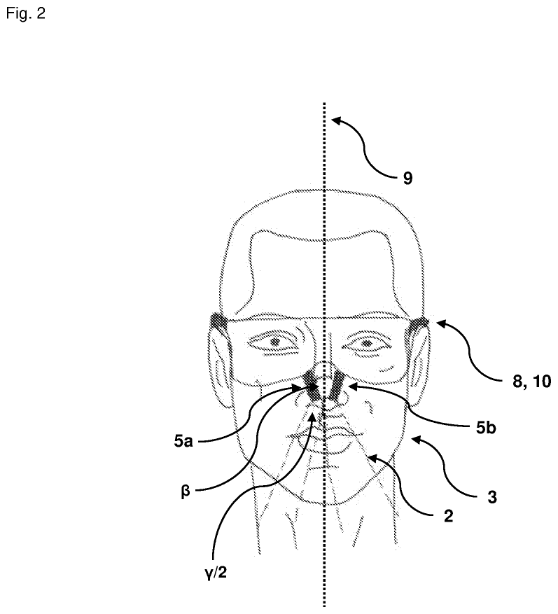

FIG. 2 shows a front view of a preferred configuration of the proposed protection device (1). The preferred embodiment of the protection device (1) which is illustrated in FIG. 2 is designed as safety goggles (8), wherein the nozzle assembly (5) is preferably arranged below the goggle lenses or below a transitional region between the goggle lens sections. Running through this central region of the safety goggles (8) there is preferably also a vertical imaginary axis (9), which divides the face (3) of the user into two face halves.

In particular, FIG. 2 shows a nozzle assembly (5) which comprises two individual nozzles (5a, 5b). It is possible, for example, for the first individual nozzle (5a) to be arranged on the left-hand half of the face in relation to a vertical virtual axis (9), while a second individual nozzle (5b) is situated on the right-hand half of the face of the user in relation to the vertical virtual axis (9). The two individual nozzles (5a, 5b) are of sloping design in relation to the vertical virtual axis (9), wherein the two individual nozzles (5a, 5b) are preferably arranged symmetrically with respect to an axis. In this case, the vertical virtual axis (9) can preferably act as an axis of symmetry. The slope of the two individual nozzles (5a, 5b) is manifested, for example, by the fact that the individual nozzles (5a, 5b) are at a smaller distance from the vertical virtual axis (9) in a lower region than in an upper region. The slope of the two individual nozzles (5a, 5b) is preferably described by the slope angle beta, wherein the angle beta is in a range of from 0 to 10 degrees.

The individual nozzles (5a, 5b) each emit an air curtain (2), wherein the two air curtains (2) together form the overall air curtain (2). The outer edge jets of the air curtain (2) start from the outlets of the nozzles (5) and define the air curtain (2), which is preferably of triangular design. The outer edge jets of the air curtain (2) are preferably symmetrical with respect to one another, wherein the vertical virtual axis (9) preferably acts as the axis of symmetry. The size of the air curtain (2) defined by the outer edge jets is determined by the opening angle gamma, wherein the angle gamma corresponds to the total opening angle enclosed by the two outer edge jets of the air curtain (2). The vertical virtual axis (9) preferably divides the opening angle gamma into two halves of equal size, which each amount to “gamma/2”. In other words, the “half opening angle gamma/2” extends between the vertical virtual axis (9) and each of the outer edge jets of the air curtain (2).

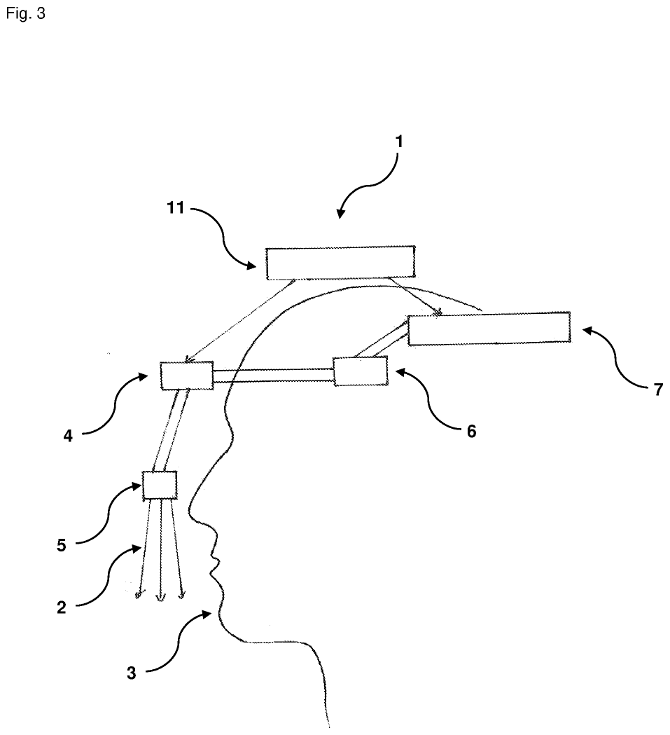

FIG. 3 shows a schematic side view of a preferred configuration of the protection device (1). The preferred embodiment of the protection device (1) which is illustrated in FIG. 3 can be designed as a protective helmet, wherein the protective helmet is designed to be worn by a worker during work. The figure illustrates a nozzle (5) or a nozzle assembly (5), which is designed to produce an air curtain (2) in front of the face (3) of a wearer of the protection device (1). Here, the air flow with which the air curtain (2) is produced preferably comes from a pump (4), which can be secured on the helmet of the protection device (1) in the preferred embodiment of the invention which is illustrated in FIG. 3. In other embodiments of the invention, it is also possible for the pump (4) to be arranged at other locations within the device (1). The pump (4) is preferably connected to the nozzle (5) or the nozzle assembly (5) by conduit means, thus enabling the air flow which is produced by the pump (4) to be passed to the nozzle (5) or the nozzle assembly (5). The conduit means can preferably be designed as air hoses. The pump (4) can furthermore be connected by conduit means to a filter (6). By means of the filter (6), the air drawn in by the pump (4) can be cleaned before it is blown into the mouth-nose region of the user as an air curtain (2). The air cleaned by the filter preferably also extends the operational life of the proposed protection device (1) since the filtering of the air protects the mechanism of the pump (4). The pump (4) is supplied with electric energy via an energy supply device (11). This is preferably a rechargeable storage battery, making it possible to dispense with the provision of a cable-bound mains connection for power supply. The energy supply device (11) and the filter (6) can preferably be connected to a function monitoring unit (7), wherein the function monitoring unit (7) is preferably designed to monitor the components of the device (1) in respect of their functionality, and optionally to control them.

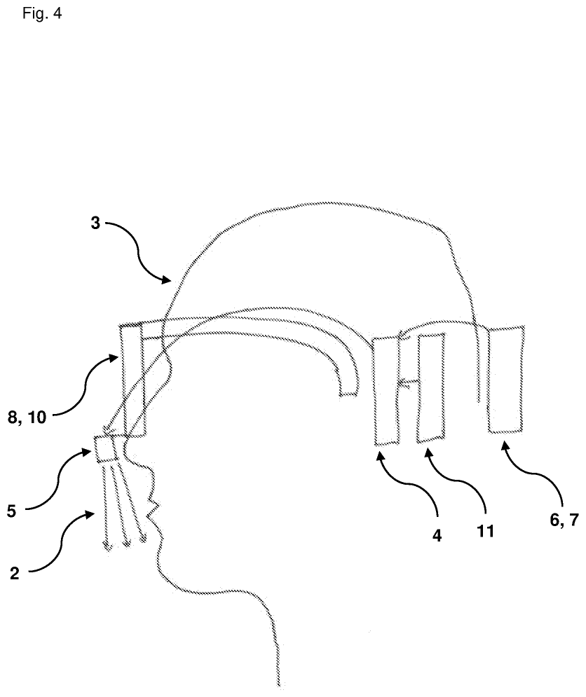

FIG. 4 shows a schematic side view of a further preferred configuration of the protection device (1). The exemplary embodiment illustrated in FIG. 4 represents safety goggles (8) which a user wears in front of their eyes. Arranged below the goggle lenses is the nozzle (5) or the nozzle assembly (5) via which the air flow for producing the air curtain (2) can be blown into the mouth-nose region of the user. Further components of the protection device (1) can be arranged on a rear side of the head of the user. If the protection device (1) is designed as safety goggles (8)—as in FIG. 4—the device (1) can comprise a belt, which is formed by an elastic band, for example, that is placed or passed around the (back of the) head of the user and there comprises the further components of the protection device (1), while the front part of the protection device (1) is formed by the safety goggles (8). The further components of the protection device (1), which can be arranged in the region of the back of the head of the user, for example, can be the pump (4), the energy supply device (11), the filter (6) and/or the function monitoring unit (7), for example.

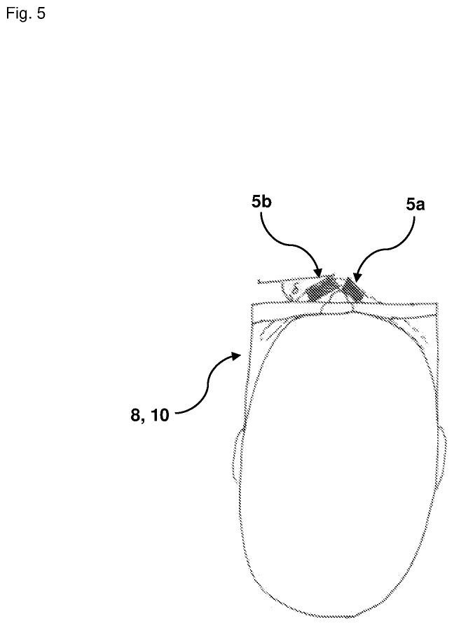

FIG. 5 shows the plan view of a preferred configuration of the protection device (1), i.e. an illustration of the invention from above. The exemplary embodiment of the protection device (1) which is illustrated in FIG. 5 is designed as safety goggles (8). The lenses of the safety goggles (8) can be used to define a vertical plane (not depicted), which is formed notionally substantially parallel to a surface of the face of the user. As illustrated in FIG. 5, the two nozzles (5a, 5b) of the nozzle assembly (5) of the illustrated exemplary embodiment of the invention can be designed to slope relative to this vertical plane and, for example, can be arranged like the arms of a letter “V”. The point of the letter “V” which is formed by the two individual nozzles (5a, 5b) preferably points away from the user, while the opening of the letter “V” faces in the direction of the face (3) of the user. The nozzles (5a, 5b) preferably follow the course of the alae of the nose of the user. The oblique positioning of the nozzles (5a, 5b) of the nozzle assembly (5) can be described by the angle delta enclosed by the vertical virtual plane and the nozzles (5a, 5b). The angles for the two nozzles (5a, 5b) preferably correspond in respect of their absolute value and are opposed as regards their sign. In other words, one nozzle (e.g. 5a) has a positive slope angle delta, while the other nozzle (e.g. 5b) has a negative slope angle delta or vice versa.

### LIST OF REFERENCE SIGNS

1 Device
2 Air curtain
3 Face
4 Pump
5 Nozzle
6 Filter
7 Function monitoring unit
8 Safety goggles
9 Vertical virtual axis
10 Supporting device
11 Energy supply device
d Distance between nozzle and end of nose

What is claimed is:

1-13. (canceled)

14: A device for producing an air curtain in front of a face of a user, the device comprising:
at least one pump for supplying air to produce the air curtain;
at least one nozzle for forming the air curtain and capable of being arranged in a region of a nose of the user on a supporting device in such a way that a mouth-nose region of the user is covered by the air curtain.

15: The device as recited in claim 14 further comprising a filter.

16: The as recited in claim 14 further comprising the supporting device, wherein the supporting device is a protective helmet or safety goggles.

17: The device as recited in claim 14 further comprising a monitor for monitoring a function of the device.

18: The device as recited in claim 14 wherein the at least one nozzle is supported at a distance d from a face of the user, wherein the distance d is in a range of 2 to 30 mm.

19. The device as recited in claim 18 wherein the distance d is 5 to 20 mm.

20: The device as recited in claim 19 wherein the distance d is 10 mm.

21: The device as recited in claim 14 wherein the at least one nozzle is designed to emit an air flow at an angle alpha, wherein the angle alpha is in a range of from 10 to 30 degrees.

22: The device as recited in claim 14 wherein the at least one nozzle includes two nozzles enclosing an angle beta with one another and wherein the angle beta is in a range of from 0 to 10 degrees.

23: The device as recited in claim 14 wherein the at least one nozzle includes two nozzles enclosing an angle delta with a vertical plane, wherein the angle delta is in a range of from 10 to 30 degrees.

24: The device as recited in claim 14 further comprising an energy supply for supplying energy for the device.

25: The device as recited in claim 14 wherein the pump is designed to produce a substantially pulsation-free air flow, with the result that a laminar flow is formed.

26: The device as recited in claim 14 wherein the pump is designed as a piezoelectric pump.

27: The device as recited in claim 14 wherein the pump operates in a frequency range of greater than 500 Hz.

28: The device as recited in claim 14 wherein the pump is designed to produce a volume flow in a range of from 1.0 to 1.5l/min.
